(19) United States
(12) Patent Application Publication
(10) Pub. No.: US 2021/0052432 A1
(43) Pub. Date: Feb. 25, 2021

(54) DRESSING

(71) Applicant: Aatru Medical, LLC, Cleveland, OH (US)

(72) Inventors: John Buan, Maple Grove, MN (US); Richard L. Middaugh, Rocky River, OH (US); Timothy Wojciechowski, Westlake, OH (US); Thomas E. Lash, Chardon, OH (US)

(21) Appl. No.: 16/547,891

(22) Filed: Aug. 22, 2019

(51) Int. Cl. A61F 13/02; A61B 46/00

(52) U.S. Cl. CPC A61F 13/0209; A61F 13/0256; A61F 2013/15569; A61F 13/0269; A61B 46/40

(57) ABSTRACT

A dressing includes a drape, a pressure-sensitive acrylic-based adhesive disposed on a skin-facing surface of the drape, an island of absorbent material including non-woven superabsorbent fibers, and a wound contact layer. The drape is a thin film capable of maintaining a negative pressure underneath the drape upon application of a vacuum. The island of absorbent material has a smaller area than the drape and is connected with and disposed beneath the sin-facing surface of the drape so as to leave a first margin of adhesive-coated drape around the island of absorbent material. The island of absorbent material further includes interruptions to reduce or eliminate tension or compression in the island of absorbent material as the dressing bends. The wound-contact layer is disposed on a skin-facing side of the island of absorbent material. The wound contact layer is configured not to stick to a wound.

### BACKGROUND

Negative pressure is a term used to describe a pressure that is below normal atmospheric pressure. Known topical negative pressure devices range from cumbersome wrinkle reducing suction apparatuses to wound therapies that include fluid-permeable wound cavity filling elements, covering dressings, reasonably air-tight means for sealing against the skin, and drainage tubes connecting the wound site and cavity filling element to the vacuum source via a fluid collection canister.

One type of dressing used with negative pressure includes a porous foam positioned on the wound and a dressing cover over the porous foam. The drainage tube connected with a pump siphons exudate from the wound after a water tight seal around the wound has been provided. Advancements can be made with regard to these dressings used with negative pressure.

### SUMMARY

In view of the foregoing, a dressing includes a drape, a pressure-sensitive acrylic-based adhesive disposed on a skin-facing surface of the drape, an island of absorbent material including non-woven superabsorbent fibers, and a wound contact layer. The drape is a thin film capable of maintaining a negative pressure underneath the drape upon application of a vacuum. The island of absorbent material has a smaller area than the drape and is connected with and disposed beneath the skin-facing surface of the drape so as to leave a first margin of adhesive-coated drape around the island of absorbent material. The island of absorbent material further includes interruptions to reduce or eliminate tension or compression in the island of absorbent material as the dressing bends. The wound-contact layer is disposed on a skin-facing side of the island of absorbent material. The wound contact layer is configured not to stick to a wound.

In the dressing mentioned above, the interruptions can be cuts extending from an upper side of the island of absorbent material through the island of absorbent material to the skin-facing side of the island of absorbent material.

In another embodiment, the interruptions can be depressions extending from an upper side of the island of absorbent material toward the skin-facing side of the island of absorbent material.

In yet another embodiment, the interruptions may be depressions extending from an upper side of the island of absorbent material toward the skin-facing side of the island of absorbent material.

In still another embodiment, the interruptions may be notches extending from an upper side of the island of absorbent material toward the skin-facing side of the island of absorbent material.

In yet another embodiment, the island of absorbent material includes a plurality of islands of absorbent material each connected with the drape and spaced from one another. The spacing between the island of absorbent material defines the interruptions.

In any of the dressings mentioned above, the interruptions are aligned transverse with a longest dimension of the drape.

In any of the dressings mentioned above, the interruptions are aligned perpendicular to a longest dimension of the drape.

In any of the dressings mentioned above, the interruptions include interruptions extending inwardly from a peripheral edge of the island of absorbent material.

In any of the dressing mentioned above, the dressing may further include a pressure indicator disposed on a skin-facing side of the drape for indicating when the dressing is under negative pressure.

In any of the dressings in the aforementioned paragraph, the pressure indicator can be configured to reveal a color element when pressed by the drape as an indicator for when the dressing is under negative pressure.

In any of the dressings mentioned above, the dressing may further include an exudate indicator disposed on skin-facing side of the drape for indicating when a level of exudate within the dressing reaches a predetermined threshold.

In any of the dressings in the aforementioned paragraph, the exudate indicator can be configured to change color to indicate when the level of exudate within the dressing has reached the predetermined threshold.

In any of the dressings mentioned above, the drape may include interruptions to relieve or eliminate tension or compression in the drape as the dressing bends.

In any of the dressings in the aforementioned paragraph, the interruptions on the drape may be slits positioned on at least one side of the drape.

In any of the dressings mentioned above, the wound contact layer may include interruptions to eliminate tension or compression in the wound contact layer as the dressing bends.

In any of the dressings in the aforementioned paragraph, the interruptions in the wound contact layer coincide with the interruptions in the island of absorbent material.

In any of the dressings mentioned above, the dressing may further include a sealing element for maintaining negative pressure underneath the dressing.

In any of the dressings in the aforementioned paragraph, the sealing element may include wedge-shaped cut-outs to relieve or eliminate tension or compression in the sealing element as the dressing bends.

Furthermore, in an embodiment, the sealing element may be a silicone gel on a backing film, which is affixed to the skin-facing surface of the drape.

In any of the dressings mentioned above, the dressing may further include a release liner having larger area than the drape. The release liner is coated with a release coating on at least one side of the release liner. The release coating is in contact with the pressure-sensitive acrylic-based adhesive in the first margin of the adhesive-coated drape and in contact with the silicone gel.

### BRIEF DESCRIPTION OF THE DRAWINGS

FIG. 1 is a schematic plan view of an example of a dressing.

FIGS. 2-5 each are a top view of an alternative dressing showing different drape and gasket configurations.

FIG. 6 is an exploded side view of the dressing depicted in FIG. 1.

FIGS. 7-14 each are a top view of an alternative dressing showing different island of absorbent material and sealing gasket configurations.

FIG. 15 is a schematic cross-sectional view of a pressure indicator.

FIG. 16 is a flow diagram depicting an example of a process of assembling the dressing depicted in FIG. 1.

FIG. 17 is a schematic cross-sectional view of the dressing placed on skin around a tissue site.

### DETAILED DESCRIPTION

The invention is not limited in its application to the details of construction and arrangement of components provided in the following description or illustrated in the attached drawings. The invention is capable of other embodiments and being practiced in various manners. The phraseology and terminology used herein is for the purpose of description and should not be regarded as limiting. Moreover, the use of “including,” “comprising,” or “having” and variations thereof is meant to encompass the items listed thereafter and equivalents thereof as well as additional items.

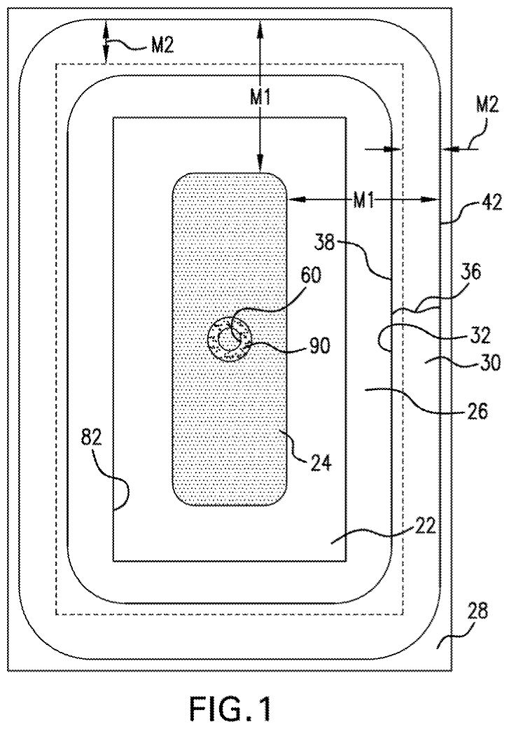

The present disclosure generally relates to negative pressure-type wound dressings, but the dressings described herein need not always be used with negative pressure. FIG. 1 depicts an embodiment of a wound dressing 20 that is useful to create a pressure that is below normal atmospheric pressure beneath the dressing 20. The dressing 20 generally includes a drape 22, an island of absorbent material 24, silicone gel 26, and a release liner 28. The arrangement of the components of the dressing 20 and the material from which the components are constructed facilitates conformity of the dressing around a wound, surgical incision, or other tissue site (hereinafter simply referred to as a “tissue site”) so as to maintain a negative pressure environment beneath the drape 22 and around the tissue site for extended periods of time, and also allows easier handling for placement of the dressing onto the skin. The dressing 20 may be made in a variety of shapes and sizes to cover a variety of tissue sites.

The drape 22 is a thin film capable of maintaining a negative pressure underneath the drape 22 upon application of a vacuum. The thin film from which the drape 22 is made can be substantially impermeable to liquids but somewhat permeable to water vapor, while still being capable of maintaining negative pressure underneath the drape 22. For example, the thin film material from which the drape 22 is made may be constructed of polyurethane or other semi-permeable material such as that sold under the Tegaderm® brand or 9834 TPU tape available from 3M. Similar films are also available from other manufacturers. Even though the film from which the drape 22 is made may have a water vapor transmission rate of about 836 g/m²/day or more, these films are still capable of maintaining negative pressure underneath the drape 22 when an appropriate seal is made around the periphery of a tissue site.

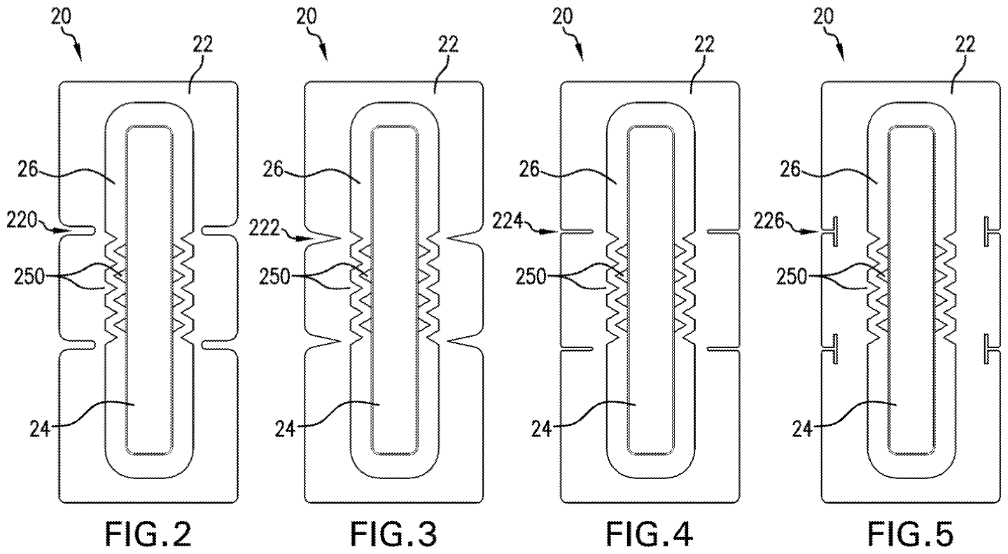

The dressing 20 can be configured to maintain negative pressure beneath the drape 22 and around the tissue site during articulating motions. As such, the dressing 20 can be disposed on a patient's knee, shin, hip, elbow, thigh, etc. to provide negative pressure therapy throughout articulating motions. However, it is to be understood that the dressing 20 can be disposed elsewhere on the patient on any region in which negative pressure therapy is to be applied. With reference to FIGS. 2-5 the drape 22 can include a plurality of interruptions positioned on the side of the drape 22 to enhance performance of the drape 22 during articulating motions. When the dressing 20 undergoes articulating motions, the interruptions relieve or eliminate tension or compression in the drape 22. As such, the interruptions allow the drape 22 to maintain contact with the skin S as the drape 22 bends throughout articulating motions. In result, negative pressure is maintained beneath the drape 22 and around the tissue site during articulating motions. With reference to FIG. 2, four interruptions are disposed on an outer periphery of a center region of the drape 22. In the embodiment illustrated in FIG. 2, the interruptions are arch-shaped cuts 220 disposed on an outer periphery of a center region of the drape 22; however, the interruptions can be any shape that effectively relieves tension or compression in the drape 22 when the dressing 20 bends during articulating motions. For example, the interruptions can be triangular shaped cuts 222, as depicted in FIG. 3. In accordance to still another embodiment depicted in FIG. 4, the interruptions are thin slits 224. FIG. 5 depicts yet another embodiment in which the interruptions are T-shaped slits 226.

With reference back to FIG. 1, the drape 22 can be cast onto a casting sheet 30, which can be made from paper. When the dressing 20 is assembled, the casting sheet 30 can be kiss cut to provide a casting sheet opening 32. The drape 22 can be made from a transparent or translucent material such that the island of absorbent material 24 and the silicone gel 26 can be visible within a “window” defined by the casting sheet opening 32 in the casting sheet 30. A cross cut 36 can extend from an internal side 38 of the casting sheet 30, after the material that defines the casting sheet opening 32 has been removed, to an outer side 42 of the casting sheet 30. Since the drape 22 is made from a very thin film, by providing a frame-like structure that is the casting sheet 30 connected with the drape 22, application of the dressing 20 is facilitated by way of providing a relatively stiffer or more rigid casting sheet 30 to grasp while placing the dressing 20 over the tissue site. After the dressing 20 has been placed, the casting sheet 30 can be grasped at the cross cut 36 and pulled towards a corner to remove the casting sheet 30 from the drape 22. As is evident in the embodiment depicted in FIG. 1, the casting sheet 30 is kiss cut around the area of the silicone gel 26 so as to allow for the person placing the dressing 20 onto the tissue site to see both the island of absorbent material 24 and the silicone gel 26 during placement of the dressing 20.

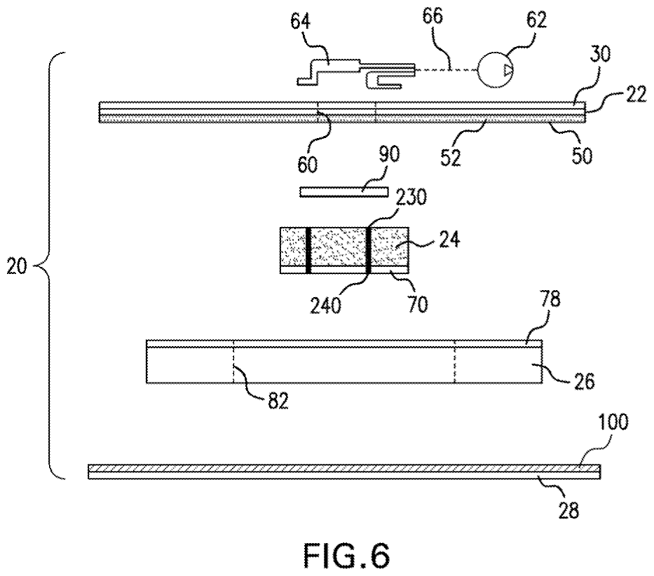

With reference to FIG. 6, a pressure-sensitive acrylic-based adhesive 50 is applied on a skin-facing surface 52 of the drape 22. Other types of adhesives could be applied to the drape 22; however, a pressure-sensitive acrylic-based adhesive is known to provide strong initial tack that can last for a relatively long time, for example a few days, when in contact with the skin. The pressure-sensitive acrylic-based adhesive 50 can be applied over an entirety of the skin-facing surface 52 of the drape 22, which can also be useful to retain other components of the dressing 20 during assembly.

The drape 22 can also include an opening 60, which can allow for the connection of a vacuum source 62 to the dressing 20. The opening 60 can be cut through the casting sheet 30 (prior to removal of the portion of the casting sheet 30 which forms the casting sheet opening 32) and the drape 22 within an area surrounded by the silicone gel 26. A fitting 64 (schematically depicted in FIG. 6) can be placed over the opening 60 and connect to the vacuum source 62 via a hose 66 (also schematically depicted in FIG. 6).

The island of absorbent material 24 is applied onto the skin-facing surface 52 of the drape 22 and is affixed to the drape 22 via the pressure-sensitive acrylic-based adhesive 50. As more clearly seen in FIG. 1, the island of absorbent material 24 has a smaller area than the drape 22 so as to leave a first margin M1 of adhesive-coated drape around the island of absorbent material 24. The first margin M1 is shown as having a vertical and horizontal component in FIG. 1, which may or may not be equal to one another. The island of absorbent material 24 can be made from a non-woven material, for example non-woven superabsorbent fibers that are pressed together. The absorbent material from which the island of absorbent material 24 is made can also be super absorbent polyester. Examples of such absorbent materials include a hydroactive wound pad available under the trademark Vilmed®. These non-woven absorbent materials are different than foam absorbent materials in that strands of fibers, e.g. strands of super absorbent polyester fibers, are pressed together to make the non-woven absorbent material.

With reference to FIG. 6, a wound contact layer 70 can be provided on a skin-contacting side of the island of absorbent material 24, if desired, which is very compatible with skin and other tissue. The wound contact layer 70 can be a perforated film that directly contacts the tissue site and is configured not to stick to the tissue site or irritate the tissue site. The perforated film can include a silicone coating, and the perforations or plurality of openings allow exudate to pass through the wound contact layer 70 and be retained in the island of absorbent material 24. Alternatively, a silicone coating can be overmolded or sprayed onto the island of absorbent material 24.

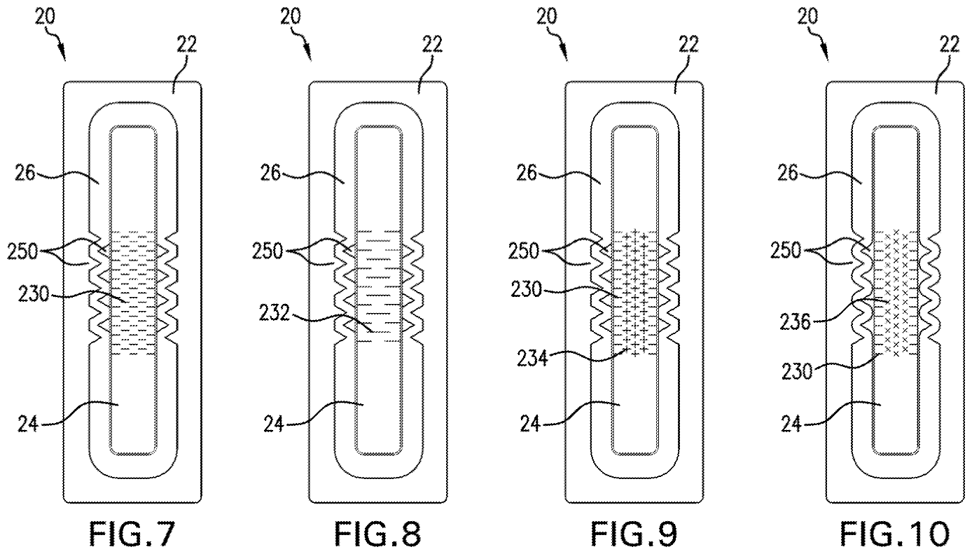

With reference to FIGS. 7-11, the island of absorbent material 24 can include a plurality of interruptions which reduce or eliminate tension or compression in the island of absorbent material 24 as the dressing 20 bends throughout articulating motions. For example, when the dressing 20 is placed on a knee, the interruptions in the island of absorbent material 24 allow the island of absorbent material 24 to continue to flex and bend as a patient bends his or her knee without any damage to the island of absorbent material 24. The interruptions can be disposed on an entirety of the island of absorbent material 24 or on only a portion of the island of absorbent material 24. The interruptions can be aligned perpendicular to the longest dimension of the drape 22. Alternatively, the interruptions can be aligned transverse with a longest dimension of the drape 22, which is intended to mean that the interruptions extend so as to cross an axis parallel with the longest dimension although the interruptions need not be aligned perpendicular to the longest dimension. Furthermore, the interruptions can include interruptions that extend inwardly from a peripheral edge of the island of absorbent material 24.

The interruptions can be, but are not limited to, slits, cuts, depressions, notches, and holes. The interruptions can extend through the island of absorbent material from a top surface to a skin-facing side of the island of absorbent material 24. Alternatively, the interruptions can be depressions or notches that extend to a portion of a height of the island of absorbent material 24 from the skin-facing side of island of absorbent material 24 or can instead extend to a portion of the height of the island of absorbent material 24 from a top surface of the island of absorbent material. Such depressions or notches can be manufactured via a calendar process that the island of absorbent material 24 may undergo prior to being fixed to the drape 22.

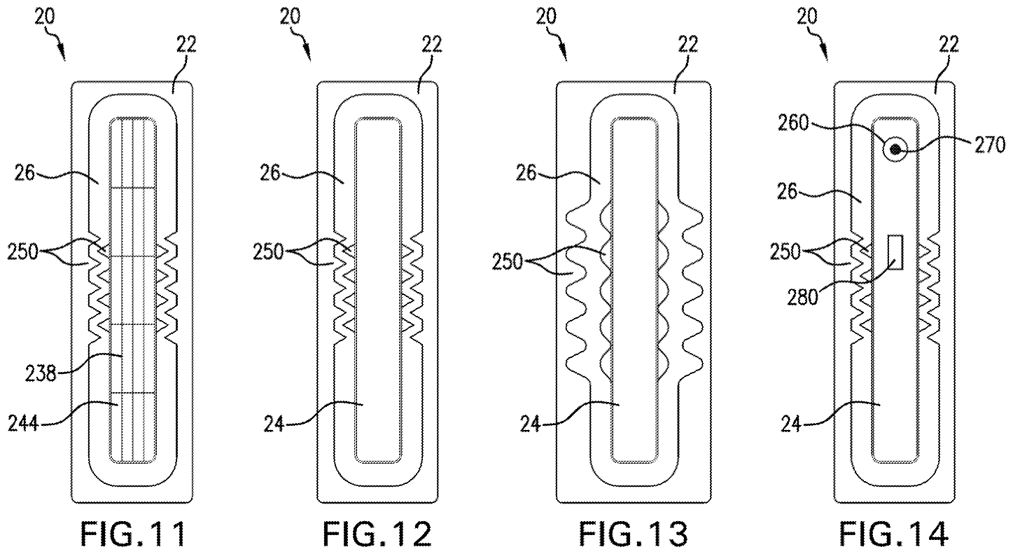

FIG. 7 depicts the interruptions as small slits 230, whereas FIG. 8 depicts the interruptions as longer slits 232. In another embodiment, the interruptions can include cross-shaped slits 234 and small slits 230, as depicted in FIG. 9. With reference to FIG. 10, the interruptions can include X-shaped slits 236 and small slits 230 in accordance to yet another embodiment. In still another embodiment, the dressing 20 comprises a plurality of islands 244 of absorbent material each connected with the drape 22 and spaced from one another, as depicted in FIG. 11. The spacing 238 between the islands 244 of absorbent materials define the interruptions.

When applied to the island of absorbent material 24, the wound contact layer 70 may cover the interruptions on the island of absorbent material 24. By positioning the wound contact layer 70 between the tissue site and the island of absorbent material 24, the dressing 20 is more comfortable for wear as the interruptions can cut through the fiber strands which may come into contact with the skin and may cause discomfort. In the embodiment in which the wound contact layer 70 is a silicone coating that is sprayed onto the island of absorbent material 24, the silicone coating can enter the bottom portions of the interruptions. Where the wound contact layer 70 is a film, after or prior to applying the wound contact layer 70 to the island of absorbent material 24, a plurality of apertures 240 (see FIG. 6) can be formed in the wound contact layer 70 that are coincident with the interruptions in the island of absorbent material 24. The apertures 240 can relieve or eliminate tension or compression in the wound contact layer 70 as the dressing 20 bends throughout articulating motions.

With reference back to FIG. 6, the silicone gel 26 is applied on a silicone gel backing film 78. The silicone gel backing film 78 can be a polyurethane, polyethylene, polypropylene, or co-polyester film that is brought in contact with the skin-facing surface 52 of the drape 22 to fix the silicone gel 26 to the drape 22. Typically, silicone does not bond well to an acrylic-based adhesive and the pressure-sensitive acrylic-based adhesive 50 is applied to the drape 22 in the illustrated embodiment. By providing the silicone gel 26 on the silicone gel backing film 78, the silicone gel 26 can be fixed with respect to the drape 22 while still being able to utilize a pressure-sensitive acrylic-based adhesive 50 on the drape 22. This allows for the benefit of providing a silicone gel 26 with the dressing 20 that can contact the skin around the tissue site and provide a much better seal than only having the pressure-sensitive acrylic-based adhesive 50, which allows for negative pressure to be obtained underneath the drape 22 around the tissue site.

The silicone gel 26 can operate as a sealing gasket for the dressing 20 to maintain negative pressure (with respect to atmosphere) underneath the dressing 20. When used for negative pressure wound therapy applications, it is desirable that the sealing gasket have the following functional characteristics: (1) the material from which the sealing gasket is made is extremely biocompatible, i.e., able to be worn for durations measured in days and weeks, with no discernible effects to the skin on which it resides, (2) the material should have mild adhesive properties, relative to skin, so that the material does not become unsealed as the wearer performs activities of daily living, and (3) the material should be flexible and conformable to adjust to the movements of the patient, while maintaining a “vacuum” seal at all times. Of the available biomedical materials, silicone gel is identified as a gasket candidate, such as the gel available from Polymer Science, Inc. as part number PS-1050. Other materials, such as hydrogel, could function as a sealing gasket but are not as biocompatible as silicone gel.

With reference to FIG. 6, the silicone gel backing film 78 has a frame shape in that a central opening 82 is provided in the silicone gel backing film 78 so that the silicone gel backing film 78 and the silicone gel 26 surrounds the island of absorbent material 24. The silicone gel backing film 78 has a smaller footprint (outer dimension) than the drape 22 and a larger footprint than the island of absorbent material 24. As such, when the silicone gel backing film 78 is affixed to the drape 22 a second margin M2 of adhesive-coated drape is positioned around the silicone gel backing film. The silicone gel 26 is positioned on the silicone gel backing film 78 and at least substantially matches the frame shape of the silicone gel backing film 78 so as to surround the island of absorbent material 24 while leaving the second margin M2 of adhesive-coated drape around the silicone gel 26. This allows the second margin M2 of adhesive-coated drape to affix to the skin around the tissue site to hold the dressing 20 in place. Since pressure-sensitive acrylic-based adhesive is not particularly suitable for an airtight or watertight seal, the silicone gel 26 provides an adequate seal so that negative pressure can be maintained within the enclosed volume 130 beneath the drape 22 and surrounded by the silicone gel 26.

The silicone gel 26 may also include interruptions for reducing or eliminating tension or compression in the silicone gel 26 as the dressing 20 bends throughout articulating motions. The interruptions permit the silicone gel 26 to maintain its seal as the silicone gel 26 bends during articulating motions. The interruptions can be in the shape of wedge-shaped cut-outs 250 disposed on a portion of the silicone gel 26 or provided along the entirety of the silicone gel 26. In the same embodiments, the wedge-shaped cut-outs 250 are disposed around the portion of the island of absorbent material 24 in which the interruptions 230, 232, 234, 236 are disposed in the island of absorbent material 24. In some embodiments, the wedge-shaped cut-outs 250 are triangular shaped, as depicted in FIG. 12. Alternatively, the wedge-shaped cut-outs 250 are more rounded, as depicted in FIGS. 10 and 13. The wedge-shaped cut-outs 250 allow the silicone gel 26 to flex when bending while also maintaining its seal around the enclosed volume 130 to maintain negative pressure underneath the dressing 20.

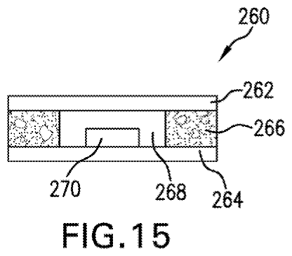

With reference to FIG. 14, a pressure indicator 260 can be disposed underneath the drape 22. With reference to FIG. 15, the pressure indicator 260 can comprise a top film 262, a bottom film 264, and an elastic element 266 interposed in-between. The elastic element 266 includes a through hole 268. In one embodiment, the elastic element 266 is a foam. A color element 270 can be positioned on a top surface of the bottom film 264 and surrounded by the elastic element 266. The pressure indicator 260 is configured to reveal the color element 270 through at least a portion of the top film 262, which is translucent or transparent, when the elastic element 266 is compressed. When the compressive force on the pressure indicator 260 is released, resilient forces in the elastic element 266 return the pressure indicator 260 to its original height. In result, the color element 270 is no longer visible through the top film 262 of the pressure indicator 260. In an embodiment, the pressure indicator 260 is disposed on the skin-facing surface 52 of the drape 22. As the pressure beneath the dressing 20 decreases, the drape 22 is drawn towards the tissue site. The pressure indicator 260 is positioned so that the drape 22 presses against the elastic element 266 when negative pressure is reached below the dressing 20. When the drape 22 compresses the pressure indicator 260, the color element 270 is visible through the top film 262 of the pressure indicator 260. In the embodiments in which the drape 22 is transparent or translucent, the color element 270 is easily visible to the user to indicate to the user that the dressing 20 is under negative pressure. When the dressing 20 is no longer under negative pressure, the drape 22 is drawn away from the pressure indicator 260 relieving its compressive force on the elastic element 266, and the color element 270 is no longer visible to the user. As such, the pressure indicator 260 can be used to indicate any substantial leaks in the dressing 20.

With reference back to FIG. 14, the dressing 20 may further comprise an exudate indicator 280. The exudate indicator 280 is configured to change colors when contacted by exudate. The exudate indicator 280 indicates to a user when a level of exudate in the dressing 20 has reached a predetermined threshold. The exudate indicator 280 can be placed anywhere in the dressing 20 to indicate when the exudate has reached the predetermined threshold while still being visible to the user. In one embodiment, the exudate indicator 280 is a strip that is placed on the skin-facing surface 52 of the drape 22 above the island of absorbent material 24. In the embodiments in which the drape 22 is transparent or translucent, the color change in the exudate indicator 280 is easily visible to the user. Thus, the exudate indicator 280 can be used as indication for when the dressing 20 needs to be replaced.

An air-permeable/liquid-impermeable filter 90 can be provided covering the opening 60 in the drape 22. As shown in FIGS. 1 and 6, the air-permeable/liquid-impermeable filter 90 is positioned against the skin-facing surface of the drape 22; however, the air-permeable/liquid-impermeable filter 90 can be provided on an outer surface of the drape 22. The air-permeable/liquid-impermeable filter 90 is made from a hydrophobic material such as expanded polytetrafluoroethylene (ePTFE). PTFE is known as being a difficult material to bond because it is “non-stick” by nature. Accordingly, other hydrophobic materials such as microporous membranes available from NXTNano LLC of Claremore, Okla., USA can be used for the air-permeable/liquid-impermeable filter 90. NXTNano LLC provides a microporous membrane constructed of nanofibers having a direct venting of 0.4 cfm while maintaining 14,000 mm hydrostatic head. The NXTNano LLC material is a thermoplastic polyurethane, which makes it easier to affix to the drape 22 via the pressure-sensitive acrylic-based adhesive 50 on the skin-facing surface 52 of the drape 22.

The release liner 28 contacts the drape 22 along the second margin M2 of the adhesive-coated drape and along with the silicone gel 26. The release liner 28 also contacts the island of absorbent material 24 and more particularly the silicone coating 70 on the island of absorbent material 24. As such, the release liner 28 should work well with both an acrylic-based adhesive and a silicone gel. Oftentimes, release liners are coated with a silicone coating; however, silicone coatings often are not compatible with silicone gel which can result in the silicone gel being pulled along with the release liner 28 when the release liner 28 is removed from the drape 22 and other components of the dressing 20.

In the illustrated example, the release liner 28 is coated with a fluorosilicone release coating 100 on the side of the release liner 28 that contacts the pressure-sensitive acrylic-based adhesive 50 on the drape 22 and the appropriate surfaces of the silicone gel 26 and the island of absorbent material 24. The release liner 28 can be a polyester film coated on one side with the fluorosilicone release coating 100, which can be used with silicone adhesives. This release coating 100 is also compatible with the pressure-sensitive acrylic-based adhesive 50 on the skin-facing surface of the drape 22 such as that available with 9834 TPU tape available from 3M. The release liner 28 has a larger area than the drape 22 and is removed from the drape 22 prior to the drape being affixed to a patient's skin around the wound site.

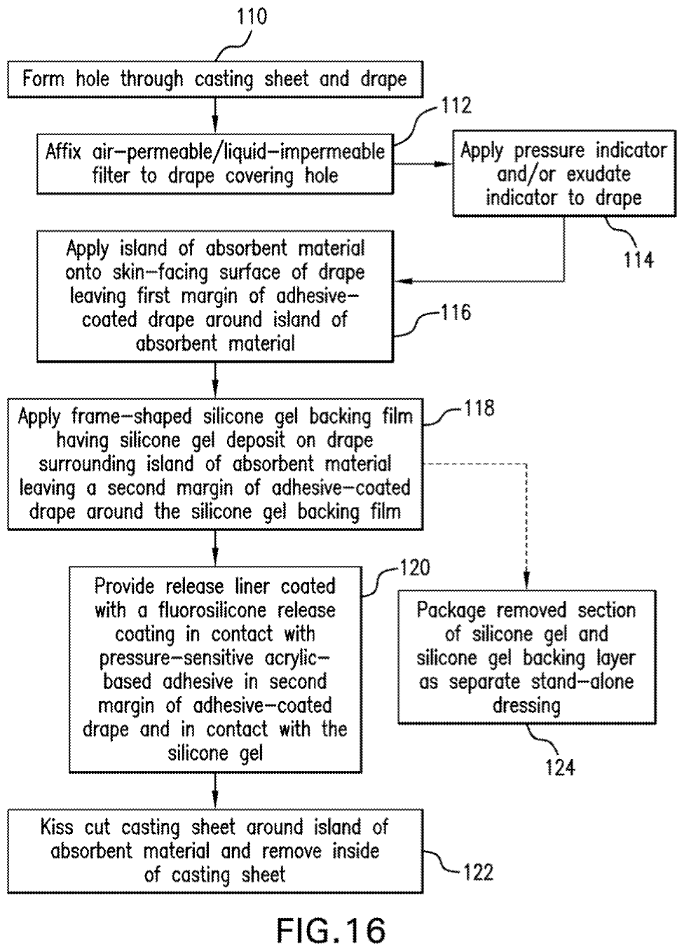

A method of assembling a dressing will be described with reference to the flow diagram shown in FIG. 16 and the embodiment of the dressing shown in FIGS. 1 and 2. The method of assembling the dressing, however, is not limited to only the embodiment of the dressing 20 shown in FIGS. 1 and 6, nor is it limited to the particular order in which the steps are described or shown in FIG. 16, unless otherwise specifically mentioned.

For example, the casting sheet 30 and the drape 22 can be provided as a pre-manufactured roll that is provided on a converter and, at 110, the opening 60 can be formed through the casting sheet 30 and the drape 22. The opening 60 can be punched or cut through the casting sheet 30 and the drape 22, or provided in another manner.

At 112, the air-permeable/liquid-impermeable filter 90 is affixed to the drape 22 covering the opening 60. The air-permeable/liquid-impermeable filter 90 can be affixed to the skin-facing surface 52 of the drape 22 via the pressure-sensitive acrylic-based adhesive 50. Alternatively, the air-permeable/liquid-impermeable filter 90 can be affixed to the surface of the drape 22 that is opposite to the skin-facing surface 52. The air-permeable/liquid impermeable filter 90 may also be omitted.

At 114, the pressure indicator 260 and/or the exudate indicator 280 are applied onto the skin-facing surface 52 of the drape 22. At 116, the island of absorbent material 24 is then applied onto the skin-facing surface 52 of the drape 22. The skin-facing surface 52 of the drape 22 has the pressure-sensitive acrylic-based adhesive 50 deposited thereon. As such, the pressure indicator 260, the exudate indicator 280, and the island of absorbent material 24 stick to the skin-facing surface 52 of the drape 22 leaving the first margin M1 of adhesive-coated drape around the island of absorbent material 24.

At 118, the silicone gel backing film 78 having the silicone gel 26 deposited thereon is applied onto the skin-facing surface 52 of the drape 22 so as to surround the island of absorbent material 24 leaving the second margin M2 of adhesive-coated drape around the silicone gel backing film 78. As discussed above, silicone does not typically adhere to an acrylic-based adhesive. As such, the silicone gel backing film 78, which can be made from a polyurethane, polyethylene, polypropylene or copolyester film, is provided having the silicone gel 26 deposited thereon so that the silicone gel 26 is fixed to the drape 22.

At 120, the release liner 28 coated with the fluorosilicone release coating 100 is provided so as to contact the pressure-sensitive acrylic-based adhesive 50 in the second margin M2 of the adhesive-coated drape and is also in contact with the silicone gel 26. The fluorosilicone release coating 100 is specially formulated so as to release from both the pressure-sensitive acrylic-based adhesive 50 and the silicone gel 26.

At 122, the casting sheet 30 is kiss cut around the island of absorbent material 24 to provide the central opening 82. An inside portion of the casting sheet 30 is removed, which forms the central opening 82 that can act as a sort of window. The drape 22 is manufactured from a transparent thin film, which allows the operator placing the dressing 20 on the tissue site the ability to view the tissue site while placing the dressing.

As mentioned above, the silicone gel 26 is applied on a silicone gel backing film 78, and the silicone gel backing film 78 has a frame shape in that the central opening 82 is provided in the silicone gel backing film 78 so that the silicone gel backing film 78 and the silicone gel 26 surround the island of absorbent material 24. To form the central opening 82, the silicone gel 26 and the silicone gel backing film 78 can be cut and removed from the remainder that makes up the frame shape. At 124, this removed section of the silicone gel 26 and the silicone gel backing film 78 can be packaged as a separate stand-alone dressing. Such a dressing can be useful to cover an incision site as an occlusive dressing that is not under negative pressure or can be used for other applications.

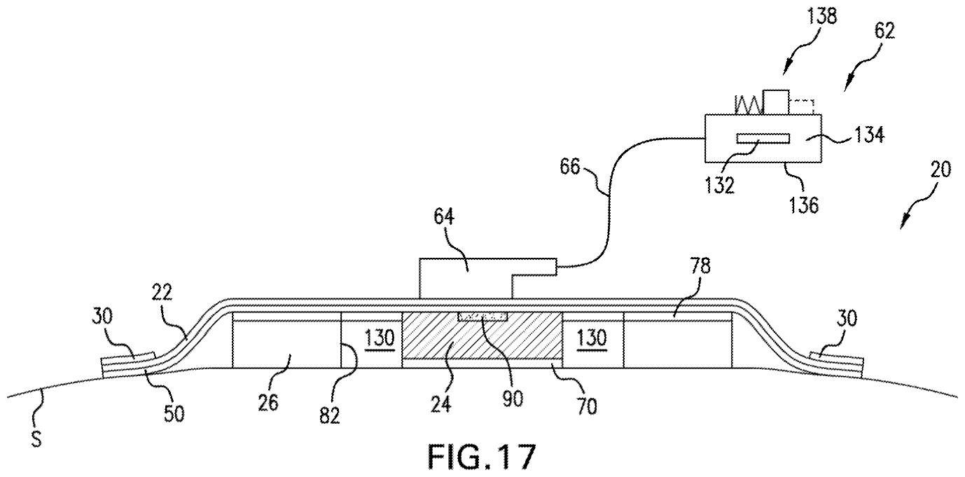

With reference to FIG. 17, in use the dressing 20 is applied to the skin S around a tissue site with the pressure-sensitive acrylic-based adhesive 50 in contact with the skin S. The drape 22 can be brought in contact with the skin S with the casting sheet 30 with the central opening 82 formed therein still attached to the drape 22. The casting sheet 30 can then be grasped near the cross cut 36 and removed from the drape 22. When the drape 22 is brought against the skin S, the silicone gel 26 surrounds the tissue site to define the enclosed volume 130, which is beneath the drape 22 and surrounded by the silicone gel 26. The vacuum source 62 is in communication with the opening 60 provided through the drape 22 to provide pneumatic communication between the vacuum source 62 and the enclosed volume 130.

The vacuum source 62 can include a reactor 132 or similar chemical pump that is configured to react with select gasses found in air to remove these gasses from air. For example, the reactor 132 can be an oxygen scavenger which removes oxygen from the air within the enclosed volume 130 so as to reduce the gas pressure within the enclosed volume by approximately 20%. Since the vacuum source 62 in this embodiment includes a chemical pump, it is important to prevent any leakage around the enclosed volume 130. The ingress of outside oxygen, which could use up the reactor 132 in the vacuum source 62, should be restricted from excessively penetrating either through the drape 22 or the silicone gel 26 or between the silicone gel 26 and the skin S. The chemical pump capacity must be sized to react with all of the oxygen within the enclosed volume 130 as well as any oxygen that permeates through the drape and/or seal during the time of use.

In FIG. 17, the reactor 132 is positioned in a closed chamber 134 that is in fluid communication with the enclosed volume 130 beneath the drape 22 and surrounded by the silicone gel 26 when the dressing 20 is affixed to skin S around the tissue site. Alternatively, the reactor 132 can be disposed in the dressing 20, making the dressing 20 a self-contained dressing. The closed chamber 134, which can be defined by a housing 136, and/or the enclosed volume 130 communicates with ambient only when a pressure differential between the closed chamber 134 and/or the enclosed volume 130 and atmosphere exceeds a predetermined threshold relative to standard atmospheric pressure. This is in contrast to known negative pressure systems which employ a mechanical pump that draws air from an enclosed volume through the mechanical pump into ambient. Leakage in these systems is not as critical since the mechanical pump typically can overcome the effect of a relatively small flow of air entering the enclosed volume by way of leakage in the system. In contrast, too much leakage when using the reactor 132 may result in the reactor 132 being consumed and no longer able to scavenge oxygen (or other gases found in air). As such, the closed chamber 134 and/or the enclosed volume 130 communicates with ambient only when the pressure differential relative to standard atmospheric pressure between the closed chamber 134 and/or the enclosed volume 130 and atmosphere exceeds a predetermined threshold, which can be between −80 mm Hg to −150 mm Hg. In another embodiment, the vacuum source can be a mechanical pump assembly, which can be provided in lieu of or can assist the reactor 132 and can also be in fluid communication with the closed chamber 134.

A dressing and a process for manufacturing a dressing have been described above with particularity. Modifications and alterations will occur to those upon reading and understanding the preceding detailed description. The invention, however, is not limited to only the embodiments described above. Instead, the invention is broadly defined by the appended claims and the equivalents thereof. It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A dressing comprising:
a drape that is a thin film capable of maintaining a negative pressure underneath the drape upon application of a vacuum;
a pressure-sensitive acrylic-based adhesive on a skin-facing surface of the drape;
an island of absorbent material comprising non-woven superabsorbent fibers, the island of absorbent material having a smaller area than the drape and being connected with and disposed beneath the skin-facing surface of the drape so as to leave a first margin of adhesive-coated drape around the island of absorbent material, wherein the island of absorbent material includes interruptions to reduce or eliminate tension or compression in the island of absorbent material as the dressing bends; and
a wound contact layer on a skin-facing side of the island of absorbent material, the wound contact layer being configured not to stick to a wound.

2. The dressing of claim 1, wherein the interruptions are cuts extending from an upper side of the island of absorbent material through the island of absorbent material to the skin-facing side of the island of absorbent material.

3. The dressing of claim 1, wherein the interruptions are depressions extending from an upper side of the island of absorbent material toward the skin-facing side of the island of absorbent material.

4. The dressing of claim 1, wherein the interruptions are notches extending from an upper side of the island of absorbent material toward the skin-facing side of the island of absorbent material.

5. The dressing of claim 1, wherein the island of absorbent material includes a plurality of islands of absorbent material each connected with the drape and spaced from one another, and spacing between the islands of absorbent material defines the interruptions.

6. The dressing of claim 1, wherein the interruptions are aligned transverse with a longest dimension of the drape.

7. The dressing of claim 1, wherein the interruptions are aligned perpendicular to a longest dimension of the drape.

8. The dressing of claim 1, wherein the interruptions include interruptions extending inwardly from a peripheral edge of the island of absorbent material.

9. The dressing of claim 1, further comprising a pressure indicator disposed on a skin-facing side of the drape for indicating when the dressing is under negative pressure.

10. The dressing of claim 9, wherein the pressure indicator is configured to reveal a color element when pressed by the drape as an indicator for when the dressing is under negative pressure.

11. The dressing of claim 1, further comprising an exudate indicator disposed on a skin-facing side of the drape for indicating when a level of exudate within the dressing reaches a predetermined threshold.

12. The dressing of claim 11, wherein the exudate indicator is configured to change color to indicate when the level of exudate within the dressing has reached the predetermined threshold.

13. The dressing of claim 1, wherein the drape includes interruptions to relieve or eliminate tension or compression in the drape as the dressing bends.

14. The dressing of claim 13, wherein the interruptions in the drape are slits positioned on at least one side of the drape.

15. The dressing of claim 1, wherein the wound contact layer includes interruptions to eliminate tension or compression in the wound contact layer as the dressing bends.

16. The dressing of claim 15, wherein the interruptions in the wound contact layer coincide with the interruptions in the island of absorbent material.

17. The dressing of claim 1, further comprising a sealing element for maintaining negative pressure underneath the dressing.

18. The dressing of claim 17, wherein the sealing element includes wedge-shaped cut-outs to relieve or eliminate tension or compression in the sealing element as the dressing bends.

19. The dressing of claim 17, wherein the sealing element is silicone gel on a backing film, which is affixed to the skin-facing surface of the drape.

20. The dressing of claim 1, further comprising a release liner having larger area than the drape, the release liner being coated with a release coating on at least one side of the release liner, the release coating being in contact with the pressure-sensitive acrylic-based adhesive in the first margin of the adhesive-coated drape and in contact with the silicone gel.
